(19) United States
(12) Patent Application Publication
(10) Pub. No.: US 2023/0191385 A1
(43) Pub. Date: Jun. 22, 2023

(54) Process for the hydroformylation of olefins using Pt and thixantphos

(71) Applicant: EVONIK OPERATIONS GMBH, Essen (DE)

(72) Inventors: Carolin Schneider, Monheim am Rhein (DE); Ralf Jackstell, Rostock (DE); Matthias Beller, Ostseebad Nienhagen (DE); Robert Franke, Marl (DE)

(73) Assignee: EVONIK OPERATIONS GMBH, Essen (DE)

(21) Appl. No.: 18/064,955

(22) Filed: Dec. 13, 2022

(30) Foreign Application Priority Data
Dec. 17, 2021 (EP) 21215381.1

(51) Int. Cl. B01J 31/30; B01J 31/24; C07F 15/00; C07C 45/50

(52) U.S. Cl. CPC B01J 31/30; B01J 31/2457; C07C 45/505; C07F 15/0086; B01J 2231/321

(57) ABSTRACT

Process for hydroformylation of olefins using Pt and thixantphos.

The present invention relates to a process for the hydroformylation of olefins using Pt and thixantphos.

P. Meessen et al., Journal of Organometallic Chemistry, 551, (1998), 165-170 describes the use of thixantphosPtCl₂ for the hydroformylation of methyl 3-pentenoate.

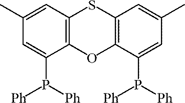

The present invention has the object of providing a novel hydroformylation process. The process should afford a good yield.

This object is achieved by a process according to Claim 1.

Process comprising the process steps of:
a) initially charging an olefin;
b) adding a compound of formula (I):

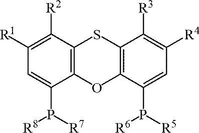

where R¹, R², R³, R⁴, R⁵, R⁶, R⁷, R⁸ are selected from: -H, -(C₁-C₁₂)-alkyl, -(C₆-C₂₀)-aryl;
c) adding a Pt compound capable of forming a complex;
d) adding a bromine compound or an iodine compound;
e) feeding in CO and H₂;
f) heating the reaction mixture from steps a) to e), to convert the olefin to an aldehyde.

In this process, process steps a) to e) can be effected in any desired sequence. Typically, however, CO and H₂ are added after the co-reactants have been initially charged in steps a) to d).

Here, process steps c) and d) may be carried out in one step by adding PtBr₂ or PtI₂.

In a preferred variant of the process, the Pt compound and the bromine compound or iodine compound are added in one step by adding PtBr₂ or PtI₂.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

Suitable (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression (C₆-C₂₀)-aryl encompasses mono- or polycyclic aromatic hydrocarbyl radicals having 6 to 20 carbon atoms. These are preferably (C₆-C₁₄)-aryl, more preferably (C₆-C₁₀)-aryl.

Suitable (C₆-C₂₀)-aryl groups are especially phenyl, naphthyl, indenyl, fluorenyl, anthracenyl, phenanthrenyl, naphthacenyl, chrysenyl, pyrenyl, coronenyl. Preferred (C₆-C₂₀)-aryl groups are phenyl, naphthyl and anthracenyl.

In one variant of the process, R⁵, R⁶, R⁷, R⁸ are -(C₆-C₂₀)-aryl.

In one variant of the process, R⁵, R⁶, R⁷, R⁸ are -Ph.

In one variant of the process, R¹ and R⁴ are (C₁-C₁₂)-alkyl.

In one variant of the process, R¹ and R⁴ are -CH₃.

In one variant of the process, R² and R³ are -H.

In one variant of the process, the compound of formula (I) has the structure (1):

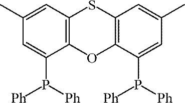

In one variant of the process, the Pt compound is selected from: Pt(II)I₂, Pt(II)Br₂, Pt(IV)I₄, Pt(IV)Br₄, diphenyl(1,5-COD)Pt(II), Pt(II)(acac)₂, Pt(0)(PPh₃)₄, Pt(0)(DVTS) solution (CAS:68478-92-2), Pt(0)(ethylene)(PPh₃)₂, Pt(II)Br₂(COD), tris(benzylideneacetone)Pt(0), Pt(II)(OAC)₂ solution, Pt(0)(t-Bu)₂, Pt(II)(COD)Me₂, Pt(II)(COD)I₂, Pt(IV)IMe₃, Pt(II)(hexafluoroacetylacetonate)₂.

In one variant of the process, the Pt compound is selected from: Pt(II)I₂, Pt(II)Br₂.

In one variant of the process, the iodine compound or the bromine compound is selected from: alkali metal halide, alkaline earth metal halide, NH₄X, alkylammonium halide, dialkyl halide, trialkyl halide, tetraalkyl halide, cycloalkylammonium halide.

In one variant of the process, in process step d), a bromine compound is added, which is Pt(II)Br₂.

In one variant of the process, the bromine compound is added in an amount in the range of 0.1 to 10, measured in equivalents based on Pt.

In one variant of the process, in process step d), an iodine compound is added, which is Pt(II)I₂.

In one variant of the process, the iodine compound is added in an amount in the range of 0.1 to 10, measured in equivalents based on Pt.

In one variant of the process, this process comprises the additional process step e′): e′) adding a solvent.

In one variant of the process, the solvent is selected from: THF, DCM, ACN, heptane, DMF, toluene, texanol, pentane, hexane, octane, isooctane, decane, dodecane, cyclohexane, benzene, xylene, marlotherm, propylene carbonate, MTBE, diglyme, triglyme, diethyl ether, dioxane, isopropanol, tert-butanol, isononanol, isobutanol, isopentanol, ethyl acetate.

In one variant of the process, the solvent is selected from: THF, DCM, ACN, heptane, DMF, toluene, texanol.

In one variant of the process, CO and H₂ is fed in at a pressure in a range from 1 MPa (10 bar) to 6 MPa (60 bar).

In one variant of the process, CO and H₂ is fed in at a pressure in a range from 2 MPa (20 bar) to 5 MPa (50 bar).

In one variant of the process, the reaction mixture is heated to a temperature in the range from 25° C. to 150° C.

In one variant of the process, the reaction mixture is heated to a temperature in the range from 30° C. to 130° C.

In one variant of the process, the olefin is selected from: ethene, propene, 1-butene, cisand/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

The invention is to be illustrated in detail hereinafter by a working example.

### EXPERIMENTAL DESCRIPTION

A vial was charged with PtX₂ (X = halogen), ligand, and an oven-dried stirrer bar. The vial is then sealed with a septum (PTFE-coated styrene-butadiene rubber) and phenolic cap. The vial is evacuated and refilled with argon three times. Toluene and 1-octene were added to the vial using a syringe. The vial was placed in an alloy plate, which was transferred to an autoclave of the 4560 series from Parr Instruments under an argon atmosphere. After purging the autoclave three times with CO/H₂, the synthesis gas pressure was increased to 40 bar at room temperature. The reaction was conducted at 80° C. for 18 h. On termination of the reaction, the autoclave was cooled to room temperature and cautiously decompressed. Yield and selectivity were determined by GC analysis.

Hydroformylation of 1-octene

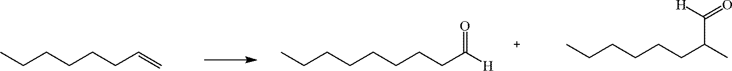

### Reaction Conditions

1.0 mmol of 1-octene, 0.5 mol% PtI₂, 2.0 equivalents of ligand, solvent: toluene, p(CO/H₂): 40 bar, T: 80° C., t: 18 h.

| | |
|---|---|
| Ligand | Yield (%) |
| 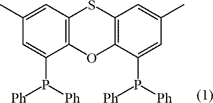 | PtI₂*: 99 |

* inventive process

### Variation of the Halogen

### Reaction Conditions

10 mmol of 1-octene, 0.1 mol% of PtX₂, 2.2 equivalents of ligand (1), solvent: toluene, p(CO/H₂): 40 bar, T: 80° C., t: 20 h.

### Yields

PtI₂: 95%
PtBr₂: 48%
PtCl₂: < 1%

As the experimental results show, the object is achieved by the inventive method.

1. Process comprising the process steps of:
a) initially charging an olefin;
b) adding a compound of formula (I): 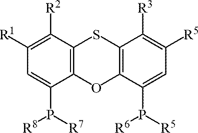
where R¹, R², R³, R⁴, R⁵, R⁶, R⁷, R⁸ are selected from: -H, -(C₁-C₁₂)-alkyl, -(C₆-C₂₀)-aryl;
c) adding a Pt compound capable of forming a complex;
d) adding a bromine compound or an iodine compound;
e) feeding in CO and H₂;
f) heating the reaction mixture from steps a) to e), to convert the olefin to an aldehyde.

2. Process according to claim 1,
where R⁵, R⁶, R⁷, R⁸ are -(C₆-C₂₀)-aryl.

3. Process according to claim 1,
where R⁵, R⁶, R⁷, R⁸ are -Ph.

4. Process according to claim 1,
where R¹ and R⁴ are -(C₁-C₁₂)-alkyl.

5. Process according to claim 1,
where R² and R³ are -H.

6. Process according to claim 1, wherein the compound of formula (I) has the structure (1): 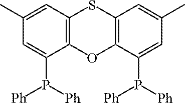
.

7. Process according to claim 1,
wherein the Pt compound is selected from: Pt(II)I₂, Pt(II)Br₂, Pt(IV)I₄, Pt(IV)Br₄, diphenyl(1,5-COD)Pt(II), Pt(II)(acac)₂, Pt(0)(PPh₃)₄, Pt(0)(DVTS) solution (CAS:68478-92-2),
Pt(0)(ethylene)(PPh₃)₂, Pt(II)Br₂(COD), tris(benzylideneacetone)Pt(0), Pt(II)(OAC)₂ solution, Pt(0)(t-Bu)₂, Pt(II)(COD)Me₂, Pt(II)(COD)I₂, Pt(IV)IMe₃, Pt(II)(hexafluoroacetylacetonate)₂.

8. Process according to claim 1,
wherein in process step d) a bromine compound is added, which is Pt(II)Br₂.

9. Process according to claim 8,
wherein the bromine compound is added in an amount in the range of 0.1 to 10, measured in equivalents based on Pt.

10. Process according to claim 1,
wherein in process step d) an iodine compound is added, which is Pt(II)I₂.

11. Process according to claim 10,
wherein the iodine compound is added in an amount in the range of 0.1 to 10, measured in equivalents based on Pt.

12. Process according to claim 1,
comprising the additional process step e′):
e′) adding a solvent.

13. Process according to claim 12,
wherein the solvent is selected from: THF, DCM, ACN, heptane, DMF, toluene, texanol, pentane, hexane, octane, isooctane, decane, dodecane, cyclohexane, benzene, xylene, marlotherm, propylene carbonate, MTBE, diglyme, triglyme, diethyl ether, dioxane, isopropanol, tert-butanol, isononanol, isobutanol, isopentanol, ethyl acetate.

14. Process according to claim 1,
wherein CO and H₂ is fed in at a pressure in a range from 1 MPa (10 bar) to 6 MPa (60 bar).

15. Process according to claim 1,
wherein the reaction mixture is heated to a temperature in the range from 25° C. to 150° C.
